(19) United States
(12) Patent Application Publication
(10) Pub. No.: US 2008/0138798 A1
(43) Pub. Date: Jun. 12, 2008

(54) Reference markers for biological samples

(76) Inventor: Greg Hampikian, Boise, ID (US)

Correspondence Address: DYKAS, SHAVER & NIPPER, LLP, P.O. BOX 877, BOISE, ID, 83701-0877, US

(21) Appl. No.: 11/024,293

(22) Filed: Dec. 23, 2004

Related U.S. Application Data
(60) Provisional application No. 60/532,673, filed on Dec. 23, 2003.

(51) Int. Cl. C12Q 1/68; C07H 21/00

(52) U.S. Cl. 435/6; 536/23.1

(57) ABSTRACT

The present invention provides methods, compositions and kits that include reference markers in biological samples. The reference samples can be marked with DNA oligomers that can be derived from sequences that do not to exist in the human genome. These sequences can be determined by an algorithm used to search published genomes for the shortest sequences which are not present (“nullomers”). Such reference markers can be used in forensic, medical, legal or other applications.

### REFERENCE MARKERS FOR BIOLOGICAL SAMPLES

This application claims priority to U.S. Provisional Patent Application No. 60/532,673, filed Dec. 23, 2003, entitled “Reference Markers for Biological Samples”.

### FIELD OF THE INVENTION

The present invention provides methods, compositions and kits that include reference markers in biological samples. The reference samples can be marked with DNA oligomers that can be derived from sequences that do not to exist in the human genome. These sequences can be determined by an algorithm used to search published genomes for the shortest sequences which are not present (“nullomers”). Such reference markers can be used in forensic, medical, legal or other applications.

### BACKGROUND TO THE INVENTION

DNA profiles are routinely used in criminal, paternity, and human identification procedures. The US military requires samples from every soldier, and every state in America requires DNA samples from convicted offenders of qualifying crimes. In addition to these targeted groups, many people are asked to give samples as victims or suspects of crimes.

Reference samples are those given by (or obtained from) known individuals who are part of a forensic, medical, legal or other identification investigation. These samples may be obtained from living or deceased individuals, or from items presumed to be derived from those individuals. A typical example is a blood sample obtained from a suspect in a criminal investigation.

Once reference samples are obtained, the integrity of the subsequent investigation and analysis depends on the integrity of the reference samples. An investigation can be completely compromised by the cross contamination of reference sample and evidentiary material. There is presently no standard marker added to blood, buccal or other biological reference samples which prevents their accidental or malicious deposition at crime scenes or on evidence samples, and it is not uncommon for the same individuals to handle reference and evidentiary samples. Should cross-contamination occur there is no reliable mechanism of demonstrating that it has happened.

Biological samples are now taken as a standard part of numerous forensic, medical, legal and identification procedures. Several states have enacted legislation defining the length of time that state agencies and forensic laboratories can hold reference samples, but others can hold these samples indefinitely. This has led to a concern on the part of those who provide the samples that errors or malicious intent could lead to their samples being mishandled, thus implicating them in criminal activity. While the DNA in a biological sample serves as an individuating identification of the donor, it says nothing of the manner in which it was obtained. The vast majority of DNA samples are taken as reference samples (known identity), and these must remain separate from evidentiary samples (unknown samples). While several patents address the labeling of samples with chemical markers, none of them satisfy the issues inherent in forensic DNA analysis.

U.S. Patent Application No. 20040072199 discloses a method for marking samples containing DNA by means of oligonucleotides. This invention does not address forensic applications, and the oligomers disclosed are artificial microsatellites and single nucleotide polymorphisms, designed without reference to avoiding sequences that might be encountered in typical forensic samples.

WO 96/17954 discloses a method for chemical identification of an object, wherein according to the invention at least two chemical markers are used. One marker shows that the container itself has been marked, while the other marker is in principle the real identification. However, such markings are not based on DNA sequences that would be readily detectable using the methodologies common in forensic, paternity and human identification laboratories.

U.S. Pat. No. 5,776,737 discloses a method for the identification of samples, wherein oligonucleotides are added to the sample obtained, which will be sequenced together with the sample after a subsequent amplification step. The oligonucleotides consist of a primer binding site and an identification region consisting of an alternating sequence of nucleotides (MN).sub.x and (MNN).sub.x, respectively, wherein N is the nucleotide of the primer binding site. The sample can be identified by sequencing the identification region. However, this method requires sequencing, and does not address the question of oligomer design in terms of avoiding sequences commonly encountered in forensic samples.

International Patent No. 20030177095 describes a system of authentication and/or tracking for identifying, tracking, authenticating and/or otherwise checking the legitimacy of one or more items which include a coded identity tag or mark, the system comprising identification means for reading said coded identity tag or mark and identifying said one or more items, storage means for storing information relating to the location, whether actual or intended, origin and/or ownership of said one or more items, and means for displaying or otherwise providing or verifying said information relating to an item when its identity tag or mark has been read. However, this system does not cover the specific application of identifying biological reference samples in order to distinguish them from evidentiary samples. It also does not embody a tag that will be identified using the standard techniques in use by forensic, medical, legal and identification laboratories namely Polymerase Chain Reaction and mitochondrial DNA sequencing. Rather it applies to a system which uses tags “preferably in the form of a coded fibre or filament” (claim 3); which can be read by a “bar code reader or scanner” (claim 4). While the claim mentions DNA in its summary (2) as a possible “tag” it does not describe any specific applications or methods using DNA as a tag.

Several patents describe forensic primer sets which are used to amplify human short tandem repeat (STR) regions of the genome. These types of techniques can be used in conjunction with the present invention. None of these patents includes a system whereby reference samples are marked with DNA tags to distinguish them from unknown, evidentiary or questioned samples.

U.S. Pat. No. 6,251,592, for example, discloses (Short Tandem Repeat) STR markers for DNA fingerprinting. This patent is a refinement on the standard technology of DNA fingerprinting for human identification using STR markers.

It is therefore an object of this invention to provide reference markers for use in biological samples that do not overlap with the information contained in the biological sample.

It is another object of the present invention to provide methods to identify reference markers for use in biological samples that do not overlap with the information contained in the biological sample.

It is further object of the present invention to provide kits for use in forensic, medical or other applications that include reference markers that do not overlap with the information contained in the biological sample.

### SUMMARY OF THE INVENTION

The present invention provides a composition including a biological sample that contains a reference oligonucleotide marker, wherein the reference oligonucleotide sequence does not overlap with a nucleotide sequence found in the genome of a living animal or organism, for example the human genome. In one embodiment the reference oligonucleotide can contain at least 11 or 12 nucleotides, which are not present in the genome of a living animal or organism. This invention also provides methods kits to produce such reference markers for use in forensic, medical, legal, and other applications.

In one embodiment, a method to produce standard reference markers (RMs) to mark and identify biological reference samples. In a second embodiment, a method is provided to add or incorporate the RMs in materials used to collect, transfer and store biological reference samples. In a third embodiment, a method to identify the RMs using techniques that are employed by laboratories involved in identification, processing and analysis of forensic, medical and legal biological reference samples.

In one aspect of the invention, oligonucleotide sequences are provided that are not found in living organisms, such as in the human genome. A method is provided to generate such sequences by searching the genomes of known organisms. The method includes an iterative search of selected data sets looking for progressively larger sequences not found in the data. Thus, the program looks first for the appearance of each two base combination, then each 3 base combination, etc. The number of possible sequences is represented by the formula 4ⁿ, where in is the length of the sequence. For an eleven base sequence, the possible number of oligomers is 4¹¹, or 4,194,304. For a 12 base sequence, there are 16,777,216 possible combinations. When the program determines that a sequence is not present in the selected data set, it records it as a nullomer.

In one embodiment, a method is provided to distinguish reference samples from those obtained as unknown, questioned or evidentiary samples. This can be achieved because the RM added to the biological reference sample, can be detected by PCR, or DNA sequencing.

In one particular embodiment, a Reference Marker (RM) nucleic acid molecule of known sequence and size can be added to reference samples as part of the collection process. The molecules can be included in the containers used to collect, transport and store these samples, such as containers for: buccal swabs, blood, other tissue, and hair samples. The RM can provide a method of distinguishing reference samples from evidentiary, questioned, and unknown samples. RMs can also provide an indicator for tampering with, misidentification of, and misinterpretation of reference samples.

The sequence of the RMs can be formulated so as not to interfere with the commonly used kits for STR analysis. Furthermore, when amplified with primers, the RMs can produce amplicons outside of the range of known human alleles produced by the STR primers in common use by legal, criminal, military, and other human identification laboratories. The RMs also can be formulated so as not to interfere with mitochondrial sequencing. RM primers can be designed in a manner known to those skilled in the art designing STR primers, wherein the primers do not amplify unintended human sequences, or produce amplicons of the reference marker size when combined with materials commonly found in crime scenes.

The RMs can be human “nullomers”. Human nullomers are small sequences which are not present in the human genome. These have been determined by an iterative search algorithm which queries sequences (downloaded to our server) for the complete set of 11, and 12 base sequence possibilities. Based on this analysis 11 and 12 nucleotide base sequences not found in the published human genome sequences can be identified. These sequences that are not found in the genome, we have given the name nullomers. These nullomers can also be searched against the entire set of known sequences in the biosphere, and those sequences that are not found in any species we have given the name “primes.” From the set of nullomers and primes, RMs and their associated primers can be designed.

The RMs can be made of DNA molecules that are either single or double stranded, synthesized oligomers or engineered fragments isolated from vectors. Other nucleotides, nucleotide analogs and organic molecules can be incorporated into the RMs so as compliment STR analysis and sequencing systems.

### DETAILED DESCRIPTION OF THE INVENTION

The current invention solves a long felt need to ensure the integrity of reference samples submitted in forensic, paternity, and other DNA tests designed to address issues of identity. The current invention offers several distinct advantages over standard methods, which include the design of non-dilutable makers suitable for forensic applications, compatibility with standard DNA identification procedures, and a built-in system for laboratory validation concerning the separation of reference and evidentiary samples.

### I: Definitions

The term “RMs” refers to reference marker which are artificial oligonucleotides added to reference samples collected from known individuals during the course of forensic, paternity, and other human identification procedures.

The term “nullomers” refers to oligonucleotide sequences of that we have determined are not present in the published genome sequences representing a single species.

The term “primes” refers to oligonucleotide sequences that have been determined are not present in any reported sequence for any species.

The term “PCR” refers to polymerase chain reaction used to amplify minute amounts of DNA. PCR is a common molecular biology technique in which cycles of denaturation, primer annealing, and primer extension with DNA polymerase, are used to multiple the number of copies of a specific sequence.

The term “amplicons” refers to the amplified products of PCR.

The term “short tandem repeat” (STR) refers to sequences between 2 and 7 nucleotides in length which are tandemly reiterated within the human organism. The STR repeats are usually reiterated between 3 and 50 times.

The term “STR profiling” refers to a length based PCR technique, which is used to identify individuals.

The term “single nucleotide polymorphism” SNP, refers to alternative nucleotide base sequences which differ by a single base. SNPs form the basis of many forms of analysis common in the art.

As used herein, the term “animal” is meant to include any non-human animal, particularly any non-human mammal, including but not limited to pigs, sheep, goats, cattle (bovine), deer, mules, horses, monkeys, dogs, cats, rats, mice, birds, chickens, C. elegans, D. melanogaster, reptiles, fish, and insects.

### II: Determination of Nullomers and Primes

Sequences publicly available on the internet at sites such as the NCBI website can be downloaded and searched using nucleotide sequences of a given length or lengths, for example, the complete set of 11 and 12 base oligomer combinations. For any species, the full set of oligomers that are not found in that species can be termed nullomers. In one embodiment of the invention, nullomers form the basis of the RM sequences to be used to mark reference samples.

In one embodiment, the nullomers described herein can be used as reference markers. In one embodiment, the nullomers can be at least 11 or 12 nucleotides in length. From these 11 or 12-mer nullomers, oligonucleotide reference markers of any size can be generated. In one embodiment, the reference markers can be at least 15 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp nucleotides in length. In another embodiment, the reference markers can be at least 70%, 75%, 80%, 85%, 90%, 95%, 97% or 99% homologous to the nullomers.

For example, for human identification RMs, the set of RMs can be derived from the set of 11 and 12 base nullomers determined for the human species. The set of 11 base nullomers derived from two published sequences of the human genome are shown in table 1. The sequences in bold represent 11 base sequences that are not found in any publicly listed sequence in the NCBI database as determined by BLAST searching on Oct. 12, 2004. These sequences that have not been reported in any species are called “Primes” (Table 2). The primes are of great value to molecular biology in that they can form the basis of an artificial DNA code, representing sequences that are not present in nature. These sequences are useful as tags indicating synthetic DNA, and the properties and novel consequences of these sequences at the DNA, RNA and protein level in engineered systems can be exploited as novel features of engineered organisms.

In a further embodiment of the invention, the list of nullomers and primes can be refined by updated searches of sequence databanks as they develop. In another embodiment, reference markers can contain a nullomer that can include any of the sequences listed in Table 1 below, or their DNA compliments. In a further embodiment, the nullomers can be at least 13 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp nucleotides in length.

TABLE 1

11 base
Nullomers
Human

| | |
|---|---|
| 1 | cgcgacgttaa |
| 2 | cgtcgctcgaa |
| 3 | tacgcgcgaca |
| 4 | cgcgcataata |
| 5 | tcgcgcgaata |
| 6 | cgcgacgcata |
| 7 | tcgacgcgata |
| 8 | tcggtacgcta |
| 9 | gcgcgacgtta |
| 10 | cgctcgacgta |
| 11 | cgacggacgta |
| 12 | tcgcgaccgta |
| 13 | gtccgagcgta |
| 14 | cgaatcgcgta |
| 15 | tgtcgcgcgta |
| 16 | cggtcgtacga |
| 17 | cgaatcgacga |
| 18 | atcgtcgacga |
| 19 | tagcgtaccga |
| 20 | gcgcgtaccga |
| 21 | cgcgtaatcga |
| 22 | ccgacgatcga |
| 23 | ctacgcgtcga |
| 24 | tatcgcgtcga |
| 25 | cgtatacgcga |
| 26 | cgattacgcga |
| 27 | tacggtcgcga |
| 28 | tattcgcgcga |
| 29 | cgatcgtgcga |
| 30 | cgattcggcga |
| 31 | cgtcgttcgac |
| 32 | tacgctcggac |
| 33 | ccgtcgaacgc |
| 34 | tcggtacgcgc |
| 35 | taacgtcgcgc |
| 36 | acgcgcgatat |
| 37 | ccgcgcgatat |
| 38 | tcgtcgacgat |
| 39 | cgacgtaccgt |
| 40 | ccgacgatcgt |
| 41 | cgaacggtcgt |
| 42 | atatcgcgcgt |
| 43 | cgacgaacggt |
| 44 | cgcgtatcggt |
| 45 | tcgacgcgtag |
| 46 | cgacgaacgag |
| 47 | cgcgtaatacg |
| 48 | cgcgctatacg |
| 49 | tcgcgtatacg |
| 50 | cgaccgatacg |
| 51 | gtcgaacgacg |
| 52 | ttcgagcgacg |
| 53 | tcgtacgaccg |
| 54 | tcgcgtaatcg |
| 55 | tcgccgaatcg |
| 56 | tcgcacgatcg |
| 57 | tcgtcgattcg |
| 58 | tacgcgattcg |
| 59 | acgaccgttcg |
| 60 | ccgatcgtcg |
| 61 | ccgttacgtcg |
| 62 | acggtacgtcg |
| 63 | tacgtccgtcg |
| 64 | accgttcgtcg |
| 65 | ctcgttcgtcg |
| 66 | cgtatcggtcg |
| 67 | tacgtcgagcg |
| 68 | cgcgtaacgcg |
| 69 | ccgaatacgcg |
| 70 | accgatacgcg |
| 71 | cgtattacgcg |
| 72 | tcgattacgcg |
| 73 | cgcgttacgcg |
| 74 | ttaacgtcgcg |
| 75 | tatgcgtcgcg |
| 76 | cgtatagcgcg |
| 77 | catatcgcgcg |
| 78 | tattatgcgcg |
| 79 | cgcgcgatatg |
| 80 | cgacgtaacgg |
| 81 | gcgttcgacgg |
| 82 | cgacgtatcgg |
| 83 | cgcgtattcgg |
| 84 | acgatcgtcgg |
| 85 | tcgatcgtcgg |
| 86 | atatcgcgcgg |

“Nullomers” not found in the human genome. “Primes” are indicated in bold.

TABLE 2
11 base “Primes”

11 Base Primes
Human nullomer #

| | |
|---|---|
| 10 | cgctcgacgta |
| 13 | gtccgagcgta |
| 32 | tacgctcggac |
| 43 | cgacgaacggt |
| 60 | ccgatacgtcg |
| 64 | accgttcgtcg |
| 82 | cgacgtatcgg |

In another embodiment, primers and probes can be synthesized that can hybridize to the oligonucleotides described herein, for example, as listed in Table 1. In a preferred embodiment, the primers hybridize under stringent conditions to these oligonucleotides. Another embodiment provides oligonucleotide probes capable of hybridizing to the oligonucleotides described herein, for example, as listed in Table 1. The polynucleotide primers or probes can have at least 14 bases, 20 bases, preferably 30 bases, or 50 bases which hybridize to a polynucleotide of the present invention. The probe or primer can be at least 14 nucleotides in length, and in a preferred embodiment, are at least 15, 20, 25, 28, or 30 nucleotides in length.

The oligonucleotides of the present invention can be synthesized by any technique known to one skilled in the art. For example, the phosphoramidite method can be used.

### III. Construction of RMs

The reference markers of the present invention can be synthesized by any technique known to one skilled in the art. In one embodiment, the primes can be used as a starting material to synthesize a longer reference marker. For example, combinations of various prime sequences can be generated that can be amplified without interfering with primers used in human identification, and without the risk of amplifying sequences commonly found in evidentiary samples, such as DNA from domestic plants and animals. Based on the sequences of nullomers and primes, RMs can be synthesized for use in conjunction with kits employed in forensic, paternity and human identification applications. Such kits are well known in the art, and are commercially available from sources such as Applied Biosystems of Foster City, Calif.

### IV. Detection of the RMs

In one embodiment, the RMs will yield amplicons of at least 10, 20, 30, 40, 50, 60, 70, 80, 90, 100 base pairs. In one embodiment the amplicon can be below the size of any common human allele used in STR profiling, for example less than 90, 80, 70, 60, 50, 40 base pairs. In another embodiment, the RM can be detected by DNA sequencing. In another embodiment, the RM can be detected by SNP analysis. In further embodiments, the RM can be identified using PCR.

PCR is based on the use of two specific synthetic oligonucleotides which are used as primers in the PCR reaction to obtain one or more DNA fragments of specific lengths. The test can detect the presence of as little as one DNA molecule per sample, giving the characteristic DNA fragment. Polymerase chain reaction (PCR): a technique in which cycles of denaturation, annealing with primer, and extension with DNA polymerase are used to amplify the number of copies of a target DNA sequence by >10⁶ times.

In general, PCR can be performed according to the following protocol (adapted from U.S. Pat. No. 4,683,195). The specific nucleic acid sequence is produced by using the nucleic acid containing that sequence as a template. If the nucleic acid contains two strands, it is necessary to separate the strands of the nucleic acid before it can be used as the template, either as a separate step or simultaneously with the synthesis of the primer extension products. This strand separation can be accomplished by any suitable denaturing method including physical, chemical or enzymatic means. One physical method of separating the strands of the nucleic acid involves heating the nucleic acid until it is completely (>99%) denatured. Typical heat denaturation can involve temperature ranging from about 80 degrees to 105 degrees Celcius for times ranging from about 1 to 10 minutes. Strand separation can also be induced by an enzyme from the class of enzymes known as helicases or the enzyme RecA, which has helicase activity and in the presence of riboATP is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67, and techniques for using RecA are reviewed in C. Radding, Ann. Rev. Genetics, 16:405-37 (1982). If the original nucleic acid constitutes the sequence to be amplified, the primer extension product(s) produced will be completely complementary to the strands of the original nucleic acid and will hybridize therewith to form a duplex of equal length strands to be separated into single-stranded molecules.

When the complementary strands of the nucleic acid or acids are separated, whether the nucleic acid was originally double or single stranded, the strands are ready to be used as a template for the synthesis of additional nucleic acid strands. This synthesis can be performed using any suitable method. Generally it occurs in a buffered aqueous solution, preferably at a pH of 7-9, most preferably about 8. Preferably, a molar excess (for cloned nucleic acid, usually about 1000:1 primer:template, and for genomic nucleic acid, usually about 10⁶:1 primer:template) of the two oligonucleotide primers is added to the buffer containing the separated template strands. It is understood, however, that the amount of complementary strand can not be known if the process herein is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP and TTP are also added to the synthesis mixture in adequate amounts and the resulting solution is heated to about 90 degrees-100 degrees Celsius for from about 1 to 10 minutes, preferably from 1 to 4 minutes. After this heating period the solution is allowed to cool to from 20 degrees-40 degrees Celsius, which is preferable for the primer hybridization. To the cooled mixture is added an agent for polymerization, and the reaction is allowed to occur under conditions known in the art. This synthesis reaction can occur at from room temperature up to a temperature above which the agent for polymerization no longer functions efficiently. Thus, for example, if DNA polymerase is used as the agent for polymerization, the temperature is generally no greater than about 45 degrees. C. An amount of dimethylsulfoxide (DMSO) can be present which is effective in detection of the signal or the temperature is 35 degrees-40 degrees Celsius. In one aspect of the invention, 5-10% by volume DMSO is present and the temperature is 35 degrees-40 degrees Celsius. For certain applications, where the sequences to be amplified are over 110 base pair fragments, an effective amount (e.g., 10% by volume) of DMSO is added to the amplification mixture, and the reaction is carried out at 35 degrees-40 degrees Celsius, to obtain detectable results or to enable cloning.

The agent for polymerization can be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There can be agents, however, which initiate synthesis at the 5′ end and proceed in the other direction, using the same process as described above.

The newly synthesized strand and its complementary nucleic acid strand form a double-stranded molecule which is used in the succeeding steps of the process. In the next step, the strands of the double-stranded molecule are separated using any of the procedures described above to provide single-stranded molecules.

New nucleic acid is synthesized on the single-stranded molecules. Additional inducing agent, nucleotides and primers can be added if necessary for the reaction to proceed under the conditions prescribed above. Again, the synthesis will be initiated at one end of the oligonucleotide primers and will proceed along the single strands of the template to produce additional nucleic acid. After this step, half of the extension product will consist of the specific nucleic acid sequence bounded by the two primers.

The steps of strand separation and extension product synthesis can be repeated as often as needed to produce the desired quantity of the specific nucleic acid sequence. As will be described in further detail below, the amount of the specific nucleic acid sequence produced will accumulate in an exponential fashion.

When it is desired to produce more than one specific nucleic acid sequence from the first nucleic acid or mixture of nucleic acids, the appropriate number of different oligonucleotide primers are utilized. For example, if two different specific nucleic acid sequences are to be produced, four primers are utilized. Two of the primers are specific for one of the specific nucleic acid sequences and the other two primers are specific for the second specific nucleic acid sequence. In this manner, each of the two different specific sequences can be produced exponentially by the present process. The polymerase chain reaction process for amplifying nucleic acid is covered by U.S. Pat. Nos. 4,683,195, 4,965,188 and 4,683,202 and European patent Nos. EP 201184 EP 200362.

DNA samples are subjected to PCR amplification using primers and thermocycling conditions specific for each locus that contains the STR of interest. In one example, the primers are selected from the group shown in Table 2. The specific amplification procedures and primer sequences relating to each locus and allelic ladder, as well as a description of locus-specific primers are described in U.S. Pat. Nos. 6,156,512 and 5,192,659.

### V. Application of RMs to Substrates and Containers

In one embodiment, the RMs can be added to a solid substrate or container. For example, the collection substrates of kits used for sample collection, such as in forensic or medical applications. Such kits are available in a number of forms and include various substrates for samples. One such product is the FTA classic card, manufactured by Whatman, plc, Brentford, Middlesex, UK. The RM molecules can be added directly to the FTA paper either during manufacture or subsequently. It can be applied as an aqueous solution, powder, gel, laminate, spray, resin, or capsule.

In another embodiment, the RMs can be added to a liquid in the collection vessel such as the Vacutainer System of Becton, Diskinson and Company, Franklin Lakes, N.J.

### VI. Kits

In other embodiments of the present inventions, kits are provided that include the oligomers and/or reference markers of the present invention. In addition, the kits can include applicator sticks, swabs, tubes, membranes, cotton, nylon, FTA paper, locking mechanisms, vessels, chambers, buffers, fixatives, drying agents, labels, bar codes, needles, microneedles, pins, lances, anticoagulants, EDTA, heparin, preservatives, primers, magnesium, DTT, dyes, antibodies, alcohol, extraction buffer, phenol, chloroform, proteinase K, SDS.

A kit containing a reference oligonucleotide marker, wherein the oligonucleotide sequence does not overlap with a nucleotide sequence in the human genome, which is deposited in or on a container. In one embodiment the kit also contains a self-locking system, wherein the swab used for buccal scraping is broken off from the applicator stick and deposited in a self sealing tube which contains the reference marker.

### EXAMPLES

The human genome has been searched using an iterative algorithm which looks for the smallest sequences not found in the selected genome. Our results are presented in Table 1 for the two publicly available human genome sequences. The oligomer sequences not found in the selected genome are called nullomers. The complete set of 11 and 12 base nullomers for the human genome have been determined using this method. These sequences can be used to construct artificial genomes, or genetic elements such as tags, novel protein epitopes, and novel RNA sequences and structures, not found in the human genome. The human nullomers were then used for BLAST searches with the goal of identifying: those sequences which were not represented in any living organism, those that were rare (represented less than 5 times in all the publicly available sequences), those not found in mammals, those not found in eukaryotes, those not found in viruses, those not found in plants, those not found in bacteria; and those not found in combinations of these organism groups. These sequences can be used to construct artificial genomes, or genetic elements such as tags, novel protein epitopes, and novel RNA sequences and structures, not found in the known sequences of the biosphere.

In one embodiment, the RMs will yield amplicons of about 60 base pairs, below the size of any common human allele found in STR profiling. One example of an RM based on nullomers is cgacgtatcgg accgttcgtcg ccgatacgtcg cgacgaacggt tacgctcggac gtccgagcgta cgctcgacgta.

1. A biological sample comprising a reference oligonucleotide marker, wherein the marker oligonucleotide sequence does not overlap with a nucleotide sequence found in the biological sample.

2. A method of identifying sequences not found in selected species (Nullomers), or those not found in any species (Primes).

3. An oligonucleotide sequence comprising a sequence selected from the group consisting of (cgcgacgttaa, cgtcgctcgaa, tacgcgcgaca, cgcgcataata, tcgcgcgaata, cgcgacgcata, tcgacgcgata, tcggtacgcta, gcgcgacgtta, cgctcgacgta, cgacggacgta, tcgcgaccgta, gtccgagcgta, cgaatcgcgta, tgtcgcgcgta, cggtcgtacga, cgaatcgacga, atcgtcgacga, tagcgtaccga, gcgcgtaccga, cgcgtaatcga, ccgacgatcga, ctacgcgtcga, tatcgcgtcga, cgtatacgcga, cgattacgcga, tacggtcgcga, tattcgcgcga, cgatcgtgcga, cgattcggcga, cgtcgttcgac, tacgctcggac, ccgtcgaacgc, tcggtacgcgc, taacgtcgcgc, acgcgcgatat, ccgcgcgatat, tcgtcgacgat, gacgtaccgt, ccgacgatcgt, cgaacggtcgt, atatcgcgcgt, cgacgaacggt, cgcgtatcggt, tcgacgcgtag, cgacgaacgag, gcgtaatacg, cgcgctatacg, tcgcgtatacg, cgaccgatacg, gtcgaacgacg, ttcgagcgacg, tcgtacgaccg, tcgcgtaatcg, tcgccgaatcg, tcgcacgatcg, tcgtcgattcg, tacgcgattcg, acgaccgttcg, ccgatacgtcg, ccgttacgtcg, acggtacgtcg, tacgtccgtcg, accgttcgtcg, ctcgttcgtcg, cgtatcggtcg, tacgtcgagcg, cgcgtaacgcg, ccgaatacgcg, accgatacgcg, cgtattacgcg, tcgattacgcg, cgcgttacgcg, ttaacgtcgcg, tatgcgtcgcg, cgtatagcgcg, catatcgcgcg, tattatgcgcg, cgcgcgatatg, cgacgtaacgg, gcgttcgacgg, cgacgtatcgg, cgcgtattcgg, acgatcgtcgg, tcgatcgtcgg, atatcgcgcgg).

4. An oligonucleotide sequence comprising a sequence selected from the group consisting of (cgctcgacgta, gtccgagcgta, tacgctcggac, cgacgaacggt, ccgatacgtcg, accgttcgtcg, cgacgtatcgg).

5. A method to collect a biological sample comprising a reference oligonucleotide marker, wherein the oligonucleotide sequence does not overlap with a nucleotide sequence found in the biological sample, and placing the biological sample in a container that also includes the reference marker.

6. The method according to any of the preceding claims, wherein the oligonucleotide markers are based on sequences found by algorithms that search for sequences not found in selected species, or those not found in any species.

7. The method according to any of the preceding claims, wherein the sample is introduced into a container containing the oligonucleotide marker.

8. The method according to any of the preceding claims, wherein polymerase chain reaction (PCR) is used to detect the markers.

9. The method according to any of the preceding claims, wherein DNA sequencing is used to detect the markers.

10. The method according to any of the preceding claims, wherein a fluorescent tag is added is added to the makers for detection or analysis.

11. The method according to any of the preceding claims, wherein the markers are added to paper, FTA paper, cotton, nylon, polymer, or textile material.

12. The method according to any of the preceding claims, wherein the markers are added to a solid support.

13. The method according to any of the preceding claims, wherein the markers are added to a liquid used in the sample collection process.

14. The method according to any of the preceding claims, wherein suitable primers are used to amplify the oligonucleotide marker sequences.

15. The method according to any of the preceding claims, wherein already available primers in kits used in forensic, legal and medical identification systems are used to detect the oligonucleotide reference marker sequences.

16. The method according to any of the preceding claims, wherein sealed containers which contain the oligonucleotide marker sequences are used.


SEQUENCE LISTING (86 sequences)

SEQ ID NO: 1 - DNA, 11 bp, Homo sapiens
cgcgacgttaa

SEQ ID NO: 2 - DNA, 11 bp, Homo sapiens
cgtcgctcgaa

SEQ ID NO: 3 - DNA, 11 bp, Homo sapiens
tacgcgcgaca

SEQ ID NO: 4 - DNA, 11 bp, Homo sapiens
cgcgcataata

SEQ ID NO: 5 - DNA, 11 bp, Homo sapiens
tcgcgcgaata

SEQ ID NO: 6 - DNA, 11 bp, Homo sapiens
cgcgacgcata

SEQ ID NO: 7 - DNA, 11 bp, Homo sapiens
tcgacgcgata

SEQ ID NO: 8 - DNA, 11 bp, Homo sapiens
tcggtacgcta

SEQ ID NO: 9 - DNA, 11 bp, Homo sapiens
gcgcgacgtta

SEQ ID NO: 10 - DNA, 11 bp, Homo sapiens
cgctcgacgta

SEQ ID NO: 11 - DNA, 11 bp, Homo sapiens
cgacggacgta

SEQ ID NO: 12 - DNA, 11 bp, Homo sapiens
tcgcgaccgta

SEQ ID NO: 13 - DNA, 11 bp, Homo sapiens
gtccgagcgta

SEQ ID NO: 14 - DNA, 11 bp, Homo sapiens
cgaatcgcgta

SEQ ID NO: 15 - DNA, 11 bp, Homo sapiens
tgtcgcgcgta

SEQ ID NO: 16 - DNA, 11 bp, Homo sapiens
cggtcgtacga

SEQ ID NO: 17 - DNA, 11 bp, Homo sapiens
cgaatcgacga

SEQ ID NO: 18 - DNA, 11 bp, Homo sapiens
atcgtcgacga

SEQ ID NO: 19 - DNA, 11 bp, Homo sapiens
tagcgtaccga

SEQ ID NO: 20 - DNA, 11 bp, Homo sapiens
gcgcgtaccga

SEQ ID NO: 21 - DNA, 11 bp, Homo sapiens
cgcgtaatcga

SEQ ID NO: 22 - DNA, 11 bp, Homo sapiens
ccgacgatcga

SEQ ID NO: 23 - DNA, 11 bp, Homo sapiens
ctacgcgtcga

SEQ ID NO: 24 - DNA, 11 bp, Homo sapiens
tatcgcgtcga

SEQ ID NO: 25 - DNA, 11 bp, Homo sapiens
cgtatacgcga

SEQ ID NO: 26 - DNA, 11 bp, Homo sapiens
cgattacgcga

SEQ ID NO: 27 - DNA, 11 bp, Homo sapiens
tacggtcgcga

SEQ ID NO: 28 - DNA, 11 bp, Homo sapiens
tattcgcgcga

SEQ ID NO: 29 - DNA, 11 bp, Homo sapiens
cgatcgtgcga

SEQ ID NO: 30 - DNA, 11 bp, Homo sapiens
cgattcggcga

SEQ ID NO: 31 - DNA, 11 bp, Homo sapiens
cgtcgttcgac

SEQ ID NO: 32 - DNA, 11 bp, Homo sapiens
tacgctcggac

SEQ ID NO: 33 - DNA, 11 bp, Homo sapiens
ccgtcgaacgc

SEQ ID NO: 34 - DNA, 11 bp, Homo sapiens
tcggtacgcgc

SEQ ID NO: 35 - DNA, 11 bp, Homo sapiens
taacgtcgcgc

SEQ ID NO: 36 - DNA, 11 bp, Homo sapiens
acgcgcgatat

SEQ ID NO: 37 - DNA, 11 bp, Homo sapiens
ccgcgcgatat

SEQ ID NO: 38 - DNA, 11 bp, Homo sapiens
tcgtcgacgat

SEQ ID NO: 39 - DNA, 11 bp, Homo sapiens
cgacgtaccgt

SEQ ID NO: 40 - DNA, 11 bp, Homo sapiens
ccgacgatcgt

SEQ ID NO: 41 - DNA, 11 bp, Homo sapiens
cgaacggtcgt

SEQ ID NO: 42 - DNA, 11 bp, Homo sapiens
atatcgcgcgt

SEQ ID NO: 43 - DNA, 11 bp, Homo sapiens
cgacgaacggt

SEQ ID NO: 44 - DNA, 11 bp, Homo sapiens
cgcgtatcggt

SEQ ID NO: 45 - DNA, 11 bp, Homo sapiens
tcgacgcgtag

SEQ ID NO: 46 - DNA, 11 bp, Homo sapiens
cgacgaacgag

SEQ ID NO: 47 - DNA, 11 bp, Homo sapiens
cgcgtaatacg

SEQ ID NO: 48 - DNA, 11 bp, Homo sapiens
cgcgctatacg

SEQ ID NO: 49 - DNA, 11 bp, Homo sapiens
tcgcgtatacg

SEQ ID NO: 50 - DNA, 11 bp, Homo sapiens
cgaccgatacg

SEQ ID NO: 51 - DNA, 11 bp, Homo sapiens
gtcgaacgacg

SEQ ID NO: 52 - DNA, 11 bp, Homo sapiens
ttcgagcgacg

SEQ ID NO: 53 - DNA, 11 bp, Homo sapiens
tcgtacgaccg

SEQ ID NO: 54 - DNA, 11 bp, Homo sapiens
tcgcgtaatcg

SEQ ID NO: 55 - DNA, 11 bp, Homo sapiens
tcgccgaatcg

SEQ ID NO: 56 - DNA, 11 bp, Homo sapiens
tcgcacgatcg

SEQ ID NO: 57 - DNA, 11 bp, Homo sapiens
tcgtcgattcg

SEQ ID NO: 58 - DNA, 11 bp, Homo sapiens
tacgcgattcg

SEQ ID NO: 59 - DNA, 11 bp, Homo sapiens
acgaccgttcg

SEQ ID NO: 60 - DNA, 11 bp, Homo sapiens
ccgatacgtcg

SEQ ID NO: 61 - DNA, 11 bp, Homo sapiens
ccgttacgtcg

SEQ ID NO: 62 - DNA, 11 bp, Homo sapiens
acggtacgtcg

SEQ ID NO: 63 - DNA, 11 bp, Homo sapiens
tacgtccgtcg

SEQ ID NO: 64 - DNA, 11 bp, Homo sapiens
accgttcgtcg

SEQ ID NO: 65 - DNA, 11 bp, Homo sapiens
ctcgttcgtcg

SEQ ID NO: 66 - DNA, 11 bp, Homo sapiens
cgtatcggtcg

SEQ ID NO: 67 - DNA, 11 bp, Homo sapiens
tacgtcgagcg

SEQ ID NO: 68 - DNA, 11 bp, Homo sapiens
cgcgtaacgcg

SEQ ID NO: 69 - DNA, 11 bp, Homo sapiens
ccgaatacgcg

SEQ ID NO: 70 - DNA, 11 bp, Homo sapiens
accgatacgcg

SEQ ID NO: 71 - DNA, 11 bp, Homo sapiens
cgtattacgcg

SEQ ID NO: 72 - DNA, 11 bp, Homo sapiens
tcgattacgcg

SEQ ID NO: 73 - DNA, 11 bp, Homo sapiens
cgcgttacgcg

SEQ ID NO: 74 - DNA, 11 bp, Homo sapiens
ttaacgtcgcg

SEQ ID NO: 75 - DNA, 11 bp, Homo sapiens
tatgcgtcgcg

SEQ ID NO: 76 - DNA, 11 bp, Homo sapiens
cgtatagcgcg

SEQ ID NO: 77 - DNA, 11 bp, Homo sapiens
catatcgcgcg

SEQ ID NO: 78 - DNA, 11 bp, Homo sapiens
tattatgcgcg

SEQ ID NO: 79 - DNA, 11 bp, Homo sapiens
cgcgcgatatg

SEQ ID NO: 80 - DNA, 11 bp, Homo sapiens
cgacgtaacgg

SEQ ID NO: 81 - DNA, 11 bp, Homo sapiens
gcgttcgacgg

SEQ ID NO: 82 - DNA, 11 bp, Homo sapiens
cgacgtatcgg

SEQ ID NO: 83 - DNA, 11 bp, Homo sapiens
cgcgtattcgg

SEQ ID NO: 84 - DNA, 11 bp, Homo sapiens
acgatcgtcgg

SEQ ID NO: 85 - DNA, 11 bp, Homo sapiens
tcgatcgtcgg

SEQ ID NO: 86 - DNA, 11 bp, Homo sapiens
atatcgcgcgg